(19) United States
(12) Patent Application Publication
(10) Pub. No.: US 2008/0152709 A1
(43) Pub. Date: Jun. 26, 2008

(54) CLONIDINE COMPOSITION AND METHOD OF USE

(75) Inventor: Jonathan David Bortz, Saint Louis, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 11/959,879

(22) Filed: Dec. 19, 2007

Related U.S. Application Data
(60) Provisional application No. 60/871,559, filed on Dec. 22, 2006.

(51) Int. Cl. A61K 31/4168; A61P 9/00; A61P 13/12; A61K 9/22; A61K 9/14; A61K 9/52; A61K 9/24; A61P 3/10; A61P 25/00

(52) U.S. Cl. 424/457; 514/392; 424/468; 424/472; 424/490

(57) ABSTRACT

A pharmaceutical composition comprises clonidine or a pharmaceutically acceptable salt or prodrug thereof. The composition, when administered to a patient in an amount delivering a clonidine dose of about 0.1 to about 2 mg/day, exhibits clonidine release properties providing a 24-hour profile of plasma clonidine concentration that (a) does not substantially or protractedly fall below about 0.2 ng/ml and exhibits a peak concentration that is therapeutically effective and does not cause unacceptable side effects in the patient; and/or (b) exhibits a peak that substantially coincides with or closely anticipates a time of maximum plasma concentration of a catecholamine occurring in a diurnal cycle of a patient having a catecholamine-mediated disease or disorder. A method for treating a disease or disorder for which clonidine is indicated in a patient comprises administering such a composition one to three times daily in a dose of about 0.1 to about 2 mg/day to the patient.

This application claims the benefit of U.S. provisional patent application Ser. No. 60/871,559, filed on Dec. 22, 2007, the entire disclosure of which is incorporated by reference herein.

### FIELD OF THE INVENTION

The present invention relates to pharmaceutical compositions comprising clonidine and to methods of use thereof, for example in treatment of catecholamine-mediated diseases and disorders of the cardiovascular system.

### BACKGROUND OF THE INVENTION

Clonidine, N-(2,6-dichlorophenyl)-4,5-dihydro-1H-imidazol-2-amine, corresponds in structure to Formula (I) below.

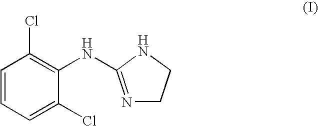

Clonidine, including its hydrochloride salt, is a well known drug effective in treatment of a wide range of clinical disorders. Clonidine is particularly useful in treatment of circulatory disorders including hypertension and cardiovascular disease related thereto, congestive heart failure and cardiomyopathy.

Clonidine is an α-adrenergic receptor agonist that exhibits affinity for central presynaptic α2 receptors in the sympathetic nervous system. These receptors are involved in control of the cardiovascular system and play a critical role in release of catecholamines such as norepinephrine by the sympathetic nervous system. The primary effect of clonidine binding to central α2 receptors is to decrease catecholamine secretion or outflow. Upon binding of clonidine to α2 receptors, a general reduction occurs in sympathetic outflow of catecholamines, for example norepinephrine, from the vasoconstrictor and cardiac accelerator centers of the medulla in the brain. The reduction in catecholamine outflow in turn leads to decreases in total peripheral resistance, renal vascular resistance, heart rate and blood pressure. Thus, clonidine is particularly effective in the treatment of hypertension and related disorders.

When administered orally, clonidine is almost completely absorbed from the gastrointestinal tract and is subject to rapid liver metabolism. A peak plasma level is generally reached within 3 to 5 hours and the plasma half-life is typically about 12 to about 16 hours, with an elimination half-life of about 6 to about 24 hours. Inter-patient variability in these parameters is rather wide.

Clonidine when administered orally has side effects including sedation and dry mouth. Severity of these side effects is dose-related, with side effects generally becoming most severe when peak plasma concentration of the drug is reached. For example, Wing et al. (1977) Eur. J. Clin. Pharmacol. 12(6):463-469 reported a linear relationship between reduction in saliva flow and plasma levels of clonidine, in a study wherein oral administration of 0.3 mg clonidine led to a peak plasma clonidine concentration of 1.34 ng/ml.

Excessively high plasma concentrations, for example above about 1.5 ng/ml, not only increase incidence and severity of side effects but lead to attenuation of the antihypertensive effectiveness of clonidine. This loss of effect at high plasma concentration may be related to a peripheral, post-synaptic α-adrenoreceptor agonist action of clonidine (see Wing et al. (1977), supra).

Davies et al. (1977) Clin. Pharmacol. Ther. 21(5):593-601 present in FIG. 3 thereof a graph showing mean plasma concentrations of clonidine in subjects following a single oral dose of 0.3 mg clonidine hydrochloride. A mean maximum concentration of 1.35 ng/ml was reached 1 hour after administration; thereafter concentration decreased exponentially to a value below 0.5 ng/ml at 24 hours. It is reported therein that all subjects in the study were markedly sedated and exhibited marked reduction in salivary flow, causing dry mouth symptoms.

Anavekar et al. (1982) Eur. J. Clin. Pharmacol. 23:1-5 measured plasma concentrations of clonidine in human subjects receiving a single oral dose of 0.075, 0.15 or 0.25 mg clonidine. Peak clonidine concentrations (Cₘₐₓ) for these doses were 0.28, 0.61 and 1.16 ng/ml respectively. By 24 hours after administration, these concentrations had fallen to 0.06, 0.07 and 0.34 ng/ml respectively.

Fujimura et al. (1994) J. Clin. Pharmacol. 34:260-265 compared pharmacokinetics of orally administered clonidine (0.075 mg twice daily for three days) and a transdermal patch formulation of clonidine. Oral administration led to a peak clonidine concentration of 0.39 ng/ml on the third day. Trough levels of clonidine in plasma at time of oral administration were in the range of about 0.1 to about 0.2 ng/ml, as shown in FIG. 2 therein. Occurrence of adverse symptoms tended to coincide with peak plasma clonidine concentrations.

On the other hand, it has been reported that as plasma concentration of clonidine falls, “wearing off” effects, including some rebound and augmentation effects, for example rebound hypertension and hyperarousal, can occur. See, for example, U.S. Pat. No. 5,484,607 to Horacek.

Subjects undergoing clonidine treatment can have individualized or unique tolerance windows of plasma concentration of clonidine characterized by a maximum peak concentration tolerated without unacceptable side-effects and/or a minimum trough concentration below which rebound and augmentation effects can occur. The “peak” and “trough” effects occurring outside the tolerance window, which in some patients can be quite narrow, can be at least inconvenient and in more severe cases clinically unacceptable, thereby limiting usefulness of oral clonidine compositions and reducing patient compliance.

Oral dosage forms said to provide sustained or delayed release of pharmaceutical actives including clonidine are proposed, for example, in the patents and publications individually cited below and incorporated herein by reference.

U.S. Pat. No. 5,133,974 to Paradissis et al.

U.S. Pat. No. 6,500,459 to Chhabra & Sarkar.

U.S. Pat. No. 6,960,357 to Chopra.

U.S. Patent Application Publication No. 2003/0050620 of Odidi & Odidi.

U.S. Patent Application Publication No. 2005/0112201 of Baichwal et al.

U.S. Patent Application Publication No. 2005/0170684 of Baichwal et al.

International Patent Publication No. WO 01/00182 of Sanofi-Synthelabo.

An extended-release dosage form of clonidine, said to have a release period of about 8 to about 12 hours and to be useful, for example, in treatment of attention deficit hyperactivity disorder, is provided in above-cited U.S. Pat. No. 5,484,607. The “peak” and “trough” effects of traditional oral clonidine formulations, such as transient sedation and rebound hyperarousal respectively, are stated therein to be overcome by use of the subject dosage form.

Steger (1980) Current Medical Research & Opinion 6(10):670-676 reported no significant difference in antihypertensive effect between a sustained-release clonidine formulation (0.3 and 0.45 mg/day) and a standard tablet (0.25 and 0.5 mg/day), but the sustained-release formulation was said to be preferred by all patients because of lesser side effects.

Macia et al. (1981) J. Cardiovasc. Pharmacol. 3(6):1193-1202 evaluated 24-hour antihypertensive efficacy of once daily oral administration of clonidine (0.25 and 0.5 mg) in a sustained-release formulation. Reduction in blood pressure was observed throughout the 24 hours and was similar during day and night.

Fyhrquist (1983) Int. J. Clin. Pharmacol. Therapy Toxicol. 21(12):634-636 reported a clinical comparison of a sustained-release depot capsule formulation of clonidine (0.25 mg once daily) and a standard formulation (0.15 mg twice daily). Both were found to be equally effective in reduction of blood pressure, but the depot form was said to be preferred by patients because of lower incidence of side effects including sedation and dry mouth.

MacGregor et al. (1985) Arzneimittelforschung 35(1A):440-446 reported pharmacokinetic data on capsule formulations of clonidine containing slow-, intermediate- or fast-dissolving tablet cores, or combinations of such cores. Peak clonidine concentrations in plasma, following oral administration of a single 0.2 mg capsule, ranged from 0.66 ng/ml (all cores fast-dissolving) to 0.46 ng/ml (all cores slow-dissolving). Time to peak ranged from 2.4 hours (all cores fast-dissolving) to 9.1 hours (all cores slow-dissolving).

Conway et al. (1992) J. Clin. Pharmacol. 32(5):427-433 conducted a comparative pharmacokinetic study of a slow-release clonidine formulation (0.15 mg once daily) and a conventional formulation (0.075 mg twice daily). Cₘₐₓ following acute administration was reportedly 0.42 ng/ml for the slow-release versus 0.70 ng/ml for the conventional formulation.

Hashimoto et al. (2003) J. Hypertens. 21(4):805-811 reported that addition to a pre-existing antihypertensive regimen of once daily administration of clonidine in the evening to patients with morning hypertension was effective in reducing morning blood pressure to levels lower than provided by the pre-existing regimen.

Transdermal administration of clonidine is widely practiced, and patch formulations for this purpose are well known. See, for example, U.S. Pat. No. 4,201,211 to Chandrasekaran et al. Administration of clonidine in a form of a patch has been shown to provide less extreme peak and trough plasma concentrations than oral administration, and is believed thereby to result in reduced incidence of “peak” and “trough” related adverse effects.

Transdermal patches, however, can cause irritation and contact dermatitis. Additionally, poor patch adherence to the skin in humid environments and in active individuals has been observed. For example, clonidine patches may need frequent replacement if a subject swims or exercises. Such inconvenience often leads to reduced compliance. A subject's failure to comply with a particular clonidine regimen can have a seriously adverse impact on success of treatment.

The incidence of many cardiovascular events follows a natural circadian or diurnal rhythm that generally reaches a maximum or peak value in the morning, typically during a period of about 2 to about 6 hours around the time of awakening from sleep. Such morning prevalence has been noted for a variety of cardiovascular events including, for example, myocardial infarction, myocardial ischemia, stroke, cardiac arrest and rupture of the abdominal aorta, and is believed to be closely related to an increase in blood pressure that is known to occur in most subjects at that time of day, mediated at least in part by heightened activity of the sympathetic nervous system.

Many biological chemicals have been shown to exhibit a diurnal rhythm of secretion in the human body. For instance, it has been widely acknowledged that hormones, neurotransmitters and other compounds are released in different amounts at different times of the day, following a diurnal or circadian pattern. Of particular importance to blood pressure regulation is catecholamine secretion. Secretion of catecholamines including norepinephrine occurs in a diurnal cycle that includes a period of maximum secretion, typically occurring in the waking period and shortly thereafter. This increase in secretion is generally known as the “morning catecholamine surge.” It should be recognized, however, that some subjects exhibit maximum catecholamine secretion at other times than in the morning, and some show no pronounced surge at any time.

There is some evidence indicating that general aging is associated with an increase in sympathetic nervous activity, for example, α-adrenergic activity related to catecholamine secretion. These systemic increases in sympathetic activity in older subjects are also believed to cause or exacerbate cardiovascular conditions including, for example, hypertension and cardiac and vascular hypertrophy. Studies have concluded that the well-known morning blood pressure increase, particularly that dependent on α-adrenergic activity, is closely associated with advanced silent hypertensive cerebrovascular disease, particularly in elderly subjects. See, for example, Kario et al. (2004) Am. J. Hypertens. 17:668-675.

Coordination of medical treatment with biological rhythms, for example diurnal rhythms, has been proposed for a number of drugs and is sometimes called “chronopharmacology” or “chronotherapy.” Such coordination takes into consideration a rhythm, such as the morning catecholamine surge, in determining optimal amount and timing of administration and/or release of medication necessary to obtain desired effects of a drug while minimizing undesired side-effects.

Sustained release oral dosage forms and transdermal patches do not necessarily deliver clonidine according to a schedule that coordinates peak concentrations of the drug with peak levels of catecholamines or with periods of elevated blood pressure.

Jain et al. (1977) Clin. Pharmacol. Ther. 21(4):382-387 proposed administering clonidine twice daily with a larger dose in the evening and a smaller dose in the morning, to limit unwanted drowsiness during the day.

Masotti et al. (1981) Int. J. Clin. Pharmacol. Res. 1/3:209-216 reported that administration of clonidine “rhythmically” according to a patient's blood pressure biorhythm was more effective than b.i.d. administration in preventing hypertensive peaks and resulted in lower incidence of side effects.

Yegnarayan & Balwani (1994) Biological Rhythm Research 25(3):329-340 reported that 0.1 mg clonidine reduced diastolic blood pressure when administered at 6 am or 12 noon but not at 4 pm or 8 pm.

Oral dosage forms said to be suitable for chronotherapy with pharmaceutical actives including clonidine are proposed in the patents and publications individually cited below and incorporated herein by reference.

U.S. Patent Application Publication No. 2003/0082230 of Baichwal et al.

U.S. Patent Application Publication No. 2004/0022852 of Chopra.

U.S. Patent Application Publication No. 2005/0118267 of Baichwal et al.

It has been proposed in U.S. Patent Application Publication No. 2002/0132001 of Garthwaite & Mathur to administer a composition comprising a delayed-release formulation of the antihypertensive drug eplerenone (an aldosterone receptor antagonist) about 6 to about 12 hours prior to the diurnal maximum concentration of aldosterone in plasma, to provide a peak eplerenone concentration in plasma that corresponds substantially to the peak aldosterone level, which typically occurs in the morning hours. It is further proposed therein that a second antihypertensive drug can be present in the composition. Clonidine, at a dose of 0.2 to 0.8 mg/day, is illustratively mentioned among many other antihypertensives.

There remains a need for methods for treating a variety of diseases or disorders using clonidine in a way that minimizes “peak” and “trough” effects, more particularly providing clonidine in amounts effective to decrease catecholamine secretion coincident with or in anticipation of a diurnal catecholamine surge. There is also a need for orally deliverable pharmaceutical compositions useful in such methods.

### SUMMARY OF THE INVENTION

There is now provided a method for treating a disease or disorder for which clonidine is indicated in a patient, comprising orally administering clonidine once daily in a dose of about 0.1 to about 2 mg to the patient in a form of a pharmaceutical composition comprising clonidine or a pharmaceutically acceptable salt or prodrug thereof and at least one pharmaceutically acceptable excipient. The composition used according to this method exhibits clonidine release properties providing, when administered at a similar dose to a plurality of test subjects, a 24-hour profile of plasma clonidine concentration, averaged over the test subjects, that does not substantially or protractedly fall below about 0.2 ng/ml and does not substantially or protractedly exceed about 1 ng/ml.

In some embodiments of the above method, the disease or disorder is mediated by a catecholamine and the patient exhibits a diurnal cycle of plasma concentration of the catecholamine having at least one diurnal peak. In such embodiments a daily administration time is identified for the composition, and the 24-hour plasma clonidine concentration profile, averaged over the test subjects, exhibits a peak that, when the composition is administered at the identified time, substantially coincides with or closely anticipates the at least one diurnal peak in plasma catecholamine concentration in the patient.

There is further provided a method for treating a disease or disorder for which clonidine is indicated in a patient, comprising
(1) establishing, for the patient, at least one of (a) a maximum plasma concentration of clonidine associated with an unacceptable side effect and (b) a minimum plasma concentration of clonidine associated with an unacceptable rebound or augmentation effect; and
(2) administering clonidine one to three times daily in a dose of about 0.1 to about 2 mg/day to the patient in a form of a pharmaceutical composition comprising clonidine or a pharmaceutically acceptable salt or prodrug thereof and at least one pharmaceutically acceptable excipient; wherein the composition exhibits clonidine release properties providing, when so administered at a similar dose to a plurality of test subjects, a 24-hour profile of plasma clonidine concentration, averaged over the test subjects, that does not substantially or protractedly fall below the minimum or does not substantially or protractedly exceed the maximum plasma concentration of clonidine established for the patient.

There is still further provided a method for treating a catecholamine-mediated disease or disorder in a patient exhibiting a diurnal cycle of plasma concentration of a catecholamine, the method comprising
(1) establishing for the patient a diurnal time of peak plasma concentration of a catecholamine;
(2) identifying a daily administration time for antihypertensive medication; and
(3) administering clonidine once daily at the administration time in a dose of about 0.1 to about 2 mg/day to the patient in a form of a pharmaceutical composition comprising clonidine or a pharmaceutically acceptable salt or prodrug thereof and at least one pharmaceutically acceptable excipient; wherein the composition exhibits clonidine release properties providing, when so administered at a similar administration time and similar dose to a plurality of test subjects, a 24-hour profile of plasma clonidine concentration, averaged over the test subjects, exhibiting a peak that substantially coincides with or closely anticipates the peak in plasma catecholamine concentration established for the patient.

There is still further provided a method for treating a catecholamine-mediated disease or disorder in a patient exhibiting a diurnal cycle of plasma concentration of a catecholamine, the method comprising
(1) establishing for the patient a diurnal time of peak plasma concentration or a diurnal period of sustained high plasma concentration of a catecholamine;
(2) selecting a composition comprising clonidine or a pharmaceutically acceptable salt or prodrug thereof and at least one pharmaceutically acceptable excipient, said composition exhibiting clonidine release properties providing, when administered to a plurality of test subjects, a 24-hour profile of plasma clonidine concentration having a peak or plateau; and
(3) administering the composition once daily in a clonidine dose of about 0.1 to about 2 mg/day to the patient, at a daily administration time selected such that the peak or plateau in plasma concentration of clonidine substantially coincides with or closely anticipates the peak or period of sustained high plasma concentration of catecholamine established for the patient.

There is still further provided a pharmaceutical composition comprising clonidine or a pharmaceutically acceptable salt or prodrug thereof, that when orally administered to a plurality of test subjects once daily in an amount delivering a clonidine dose of about 0.1 to about 2 mg/day, exhibits clonidine release properties providing a 24-hour profile of plasma clonidine concentration, averaged over the test subjects, that does not substantially or protractedly fall below about 0.2 ng/ml and does not substantially or protractedly exceed about 1 ng/ml.

In some embodiments, the composition comprises (a) a first formulation component comprising clonidine exhibiting a first release profile, for example an immediate release profile; and (b) a second formulation component comprising clonidine exhibiting a second release profile that is different from the first release profile, for example an extended and/or delayed release profile.

The 24-hour profile of plasma clonidine concentration provided by the composition can, in some embodiments, exhibit a peak about 4 to about 16 hours after administration. Such a composition, if administered in the evening, can provide a peak plasma concentration of clonidine that substantially coincides with or closely anticipates a morning catecholamine surge.

There is still further provided an orally deliverable pharmaceutical dosage form comprising
(a) zero to about 50% by weight of particles of a first kind comprising clonidine or a pharmaceutically acceptable salt or prodrug thereof in a first amount, said particles of the first kind (i) each comprising an inert substrate having a clonidine-containing layer thereon, and (ii) exhibiting a clonidine immediate release profile; and
(b) about 50% to 100% by weight of particles of a second kind comprising clonidine or a pharmaceutically acceptable salt or prodrug thereof in a second amount, said particles of the second kind (i) each comprising a particle of the first kind, additionally having a coating that comprises a dissolution modifying system comprising a film forming agent and a plasticizer, and (ii) exhibiting a clonidine extended release profile;
   wherein the clonidine or salt or prodrug thereof is present in a total clonidine equivalent amount of about 0.1 to about 2 mg; and wherein the weight ratio of particles of the first kind to particles of the second kind, said first and second amounts, and said dissolution modifying system operate to provide, when the dosage form is orally administered one to three times daily to a plurality of test subjects, a 24-hour profile of plasma clonidine concentration, averaged over the test subjects, that does not substantially or protractedly fall below about 0.2 ng/ml and exhibits a peak concentration that does not substantially or protractedly exceed about 2.5 ng/ml.

Again, in some embodiments, the 24-hour profile of plasma clonidine concentration provided by the dosage form can exhibit a peak about 4 to about 16 hours after administration. Such a dosage form, if administered in the evening, can provide a peak plasma concentration of clonidine that substantially coincides with or closely anticipates a morning catecholamine surge.

### DETAILED DESCRIPTION

The present invention provides methods for treating diseases or disorders for which clonidine is indicated, for example in a patient experiencing or at risk of such diseases or disorders.

Clonidine has been found useful in treatment of a wide range of diseases and disorders, not all of which are known to be mediated by catecholamines or even related to α-adrenergic activity. A list of therapeutic uses of clonidine has been compiled, for example, by Fagan et al. (2006) U.S. Pharmacist 5:HS2-HS16, which is incorporated by reference herein without admission that it constitutes prior art to the present invention.

Examples of diseases or disorders for which clonidine is indicated include hypertension, arrhythmia, myocardial ischemia, atrial fibrillation, congestive heart failure, allodynia, hyperalgesia, neuropathic pain, cancer pain, cluster headache, chronic headache, migraine, postoperative pain, spinal cord injury pain, akathisia, restless legs syndrome, peripheral neuropathy, neuralgia, orofacial pain, diabetic gastroparesis, chronic memory disorders, hypertonia, hyperkinetic movement disorders, Tourette's syndrome, substance withdrawal, attention deficit hyperactivity disorder, manic states, behavioral disorders related to encephalopathy, bipolar disorder, narcolepsy, post-traumatic stress disorder, schizophrenia, sleep disorders, social phobia, hyperthyroidism, growth delay, excessive sweating, hot flashes, trichorrhexis nodosa, and combinations thereof.

More particularly, methods of the invention are useful in treatment of diseases or disorders that are mediated by one or more catecholamines, for example norepinephrine, epinephrine, dopamine or an active derivative of any of these.

In some embodiments, the disease or disorder is catecholamine-mediated hypertension or a condition or event arising therefrom, which can be mediated by one or more catecholamines acting centrally on vasoconstrictor and/or accelerator centers, and/or peripherally on pre-synaptic, synaptic and/or post-synaptic levels. Methods of the invention can accordingly be used to reduce risk or incidence of an adverse cardiovascular event, for example hypertension, arrhythmia, myocardial infarction, myocardial ischemia, stroke, cardiac arrest, rupture of the abdominal aorta, or a combination thereof.

The terms “treat”, “treating” and “treatment” herein will be understood, except where the context demands otherwise, to embrace prophylactic administration to a patient not yet presenting symptoms of a disease or disorder, but at risk of developing the disease or disorder, as well as administration to a patient already having the disease or disorder. Treatment can address an underlying cause of the disease or disorder and/or can be palliative, i.e., act to reduce, alleviate or relieve symptoms that cause distress to the patient.

The patient herein can be of any animal, particularly mammalian, e.g., primate, species, but most typically is a human patient.

The present method comprises orally administering clonidine once daily in a dose of about 0.1 to about 2 mg, more typically about 0.1 to about 1 mg, to the patient in a form of a pharmaceutical composition comprising clonidine or a pharmaceutically acceptable salt or prodrug thereof and at least one pharmaceutically acceptable excipient.

“Orally administering” herein includes both peroral (i.e., per os) and intraoral (e.g., sublingual or buccal) administration, but emphasis of the present application is on peroral administration.

The particular daily dose selected will depend on a number of factors, including the nature and severity of the disease or disorder, the particular benefit(s) sought, the therapeutic response and side effect tolerance of the individual patient, inclusion or otherwise of other drugs in an antihypertensive regimen, etc. A patient may begin therapy on a relatively low daily dose and proceed stepwise to a higher dose which is then maintained during further therapy. Typically, for management of hypertension and cardiovascular risk associated therewith, a suitable daily dose will generally be about 0.1 to about 0.5 mg, for example about 0.1, about 0.15, about 0.2, about 0.25, about 0.3, about 0.35, about 0.4, about 0.45 or about 0.5 mg. Doses and other amounts of clonidine are expressed herein as free base equivalent.

The composition administered can take any orally deliverable form known in the pharmaceutical arts including a liquid (e.g., solution, emulsion or suspension), powder, granule, tablet, capsule, etc. In most embodiments, a discrete solid dosage form such as a tablet or soft or hard capsule is used. A liquid-filled or gel-filled capsule, as well as a solid-filled (e.g., containing powder, granules, cores or spheres) capsule, is considered a discrete solid dosage form in the present context. If desired, the daily dose of clonidine can be administered in a plurality of dosage forms, but most conveniently a single dosage form contains the full daily dose.

Clonidine is present in the composition in the form of free base, one or more salts or one or more prodrugs of clonidine, or in a combination of such forms. Suitably, clonidine in the form of its hydrochloride salt is used. The composition further comprises at least one pharmaceutically acceptable excipient as described more fully hereinbelow.

The composition to be used according to the present method has very particular requirements with respect to its clonidine release properties. These properties are defined in part herein by pharmacokinetic data for the composition that can be obtained from a plurality of test subjects according to any standard pharmacokinetic protocol. The test subjects herein are of the same species as the patient, i.e., in most embodiments, human, and typically adult. The protocol can involve a single dose or once daily dosing for several days, usually at least 3 days. A dose or doses used to provide pharmacokinetic data in test subjects should be similar to the dose desired to be administered to the patient. By “similar” in the present context is meant equal, or sufficiently close that, by reasonable interpolation or extrapolation from the data, pharmacokinetic properties for the desired dose can reasonably be estimated. Clinical pharmacokinetic data submitted as part of a submission to a regulatory agency (such as the U.S. Food and Drug Administration (FDA) or its counterparts in other countries) for purposes of obtaining an approved drug label generally meet the requirement herein.

Specifically, the composition used according to the present method provides a 24-hour profile of plasma clonidine concentration, averaged over the test subjects, that does not substantially or protractedly fall below about 0.2 ng/ml and does not substantially or protractedly exceed about 1 ng/ml. In various embodiments, the plasma clonidine concentration neither substantially nor protractedly falls below about 0.25, about 0.3, about 0.35, about 0.4, about 0.45, about 0.5, about 0.55 or about 0.6 ng/ml. In various embodiments, the plasma clonidine concentration neither substantially nor protractedly exceeds about 0.9, about 0.8, about 0.7 or about 0.6 ng/ml. With respect to the terms “substantially” and “protractedly” in the present context, the following situations are illustrative. A concentration that is within about 20%, for example within about 10%, below a stated minimum or above a stated maximum is considered not to “substantially” fall below or exceed the stated minimum or maximum respectively. A concentration that temporarily falls below a stated minimum or exceeds a stated maximum for a period of less than about 2 hours, for example less than about 1 hour, is considered not to “protractedly” fall below or exceed the stated minimum or maximum respectively. In particular embodiments, plasma clonidine concentration does not, to any degree and for any duration, fall below a stated minimum or exceed a stated maximum.

For a subject able to tolerate therapeutically effective plasma levels of clonidine higher than about 1 ng/ml, a composition can be administered providing a 24-hour profile exhibiting a peak concentration that does not substantially or protractedly exceed about 2.5 ng/ml, for example one that does not substantially or protractedly exceed about 2.25, about 2, about 1.75, about 1.5 or about 1.25 ng/ml. In such a case it is generally desirable that plasma clonidine concentrations remain within a relatively narrow range (for example not substantially or protractedly falling below about one-fifth of the peak concentration) during a 24-hour period.

In one particular embodiment, the plasma clonidine concentration, averaged over the test subjects, does not substantially or protractedly fall below about 0.2 ng/ml and does not substantially or protractedly exceed about 0.8 ng/ml.

In another particular embodiment, the plasma clonidine concentration, averaged over the test subjects, does not substantially or protractedly fall below about 0.4 ng/ml and does not substantially or protractedly exceed about 0.8 ng/ml.

The present invention is not limited to compositions or methods of use thereof exhibiting particular clonidine release parameters, as measured for example in a standard in vitro dissolution test. However, it will typically be found that certain release properties are associated with a pharmacokinetic profile wherein, with once daily administration, plasma clonidine concentration remains within a rather narrow window as described above. In particular, an immediate release composition as studied for example by Davies et al. (1977), supra, is likely, at higher doses, to provide a peak plasma concentration of clonidine that exceeds a maximum desired herein and, at lower doses, to provide a trough plasma concentration of clonidine that falls below a minimum desired herein.

For example, the composition will typically be one wherein release of clonidine in therapeutically meaningful amounts is still occurring at least about 8 hours, for example at least about 10 hours, at least about 12 hours, at least about 14 hours, at least about 16 hours, at least about 18 hours, at least about 20 hours, at least about 22 hours or at least about 24 hours, after administration. Such a composition can release clonidine more or less continuously over the entire period from shortly after administration until at least about 8 hours, for example at least about 10 hours, at least about 12 hours, at least about 14 hours, at least about 16 hours, at least about 18 hours, at least about 20 hours, at least about 22 hours or at least about 24 hours, after administration. This is an example of an extended-release or sustained-release composition of the invention. Alternatively, a composition can exhibit a period, for example lasting for about 1 hour to about 12 hours, immediately following administration when substantially no clonidine release occurs, followed by a release period. Such a composition is an example of a delayed-release or timed-release composition of the invention.

In some embodiments, the composition comprises two formulation components having different release characteristics. Thus a composition can comprise (a) a first formulation component comprising clonidine exhibiting a first release profile, and (b) a second formulation component comprising clonidine exhibiting a second release profile that is different from the first release profile.

Illustratively, the first release profile can be an immediate release profile and the second release profile an extended and/or delayed release profile.

In some embodiments of the present method, the disease or disorder is mediated by a catecholamine, for example norepinephrine, and the patient exhibits a diurnal cycle of plasma concentration of the catecholamine having at least one diurnal peak. Most commonly such a peak occurs in the morning, for example near the end of a sleep period, at or around awakening, or shortly after awakening. In some patients, however, a diurnal catecholamine peak occurs at other times, for example during a sleep period (typically during the night) or during a period of being awake (typically during the day), not necessarily at or around awakening but often within about 8 hours before or 8 hours after awakening. Some patients exhibit two catecholamine peaks, which can be similar in degree or can take the form of a primary peak at one time in the cycle and a secondary, i.e., lower, peak at another time in the cycle.

In such embodiments, the 24-hour plasma clonidine concentration profile, averaged over the test subjects, provided by the composition exhibits a peak that substantially coincides with or closely anticipates the at least one diurnal peak in plasma catecholamine concentration in the patient.

The phrase “substantially coincides with or closely anticipates” means that there is a sufficiently close correspondence in timing of the clonidine peak and the catecholamine peak to provide elevated clonidine levels in plasma at a time of day when the catecholamine-mediated disease or disorder, for example hypertension or a condition or event arising therefrom, is most pronounced and when, accordingly, the clonidine has greatest potential to be of benefit. For example, the clonidine peak can occur about 4 hours before to about 4 hours after the catecholamine peak, e.g., about 2 hours before to about 1 hour after the catecholamine peak. It will be understood that timing of these peaks cannot always be precisely established or predicted, and it is not required that there be exact coincidence of the clonidine peak with the catecholamine peak in order to obtain the benefits of the present method.

In a particular embodiment it can be useful to administer the clonidine composition about 4 to about 16 hours, for example about 6 to about 14 hours or about 8 to about 12 hours, before the at least one diurnal peak in plasma catecholamine concentration. The composition used according to this embodiment should exhibit release properties consistent with providing a peak clonidine concentration in plasma that substantially coincides with or closely anticipates the catecholamine peak. For example, such a composition administered once daily in the evening, i.e., between about 6 pm and about midnight, for example around bedtime, is adapted to control a morning catecholamine surge.

It is within the ordinary skill of pharmaceutical formulators, presented with the above pharmacokinetic and/or release properties adapted to minimize “peak” and “trough” effects and, in particular embodiments, to correspond to catecholamine peaks, to prepare compositions having such properties without undue experimentation. Known controlled release technologies can be applied, as illustrated hereinbelow.

An insight underlying the present invention is that individual patients differ significantly in their tolerance window for “peak” and “trough” effects of clonidine and, as indicated above, in their diurnal catecholamine cycles. Accordingly, the present invention enables clonidine therapy to be tailored to individual patients in a way that has not hitherto been contemplated.

One embodiment of the invention provides a method for treating a disease or disorder for which clonidine is indicated in a patient, comprising
(1) establishing, for the patient, at least one of (a) a maximum plasma concentration of clonidine associated with an unacceptable side effect and (b) a minimum plasma concentration of clonidine associated with an unacceptable rebound or augmentation effect; and
(2) administering clonidine one to three times daily in a dose of about 0.1 to about 2 mg/day, more typically about 0.1 to about 1 mg/day, to the patient in a form of a pharmaceutical composition comprising clonidine or a pharmaceutically acceptable salt or prodrug thereof and at least one pharmaceutically acceptable excipient; wherein the composition exhibits clonidine release properties providing, when so administered at a similar dose to a plurality of test subjects, a 24-hour profile of plasma clonidine concentration, averaged over the test subjects, that does not substantially or protractedly fall below the minimum or does not substantially or protractedly exceed the maximum plasma concentration of clonidine established for the patient.

Maximum and/or minimum plasma concentrations of clonidine tolerated by the patient without unacceptable rebound or augmentation effect can be established by a clinician who can relate incidence and severity of side effects and/or rebound effects to plasma clonidine levels measured by established laboratory techniques in blood samples collected from the patient following clonidine administration at one or a range of doses, for example intravenously or in a form of an immediate release oral dosage form. Upon establishing a maximum and/or minimum tolerated by the patient, a clonidine composition can be selected having release and/or pharmacokinetic properties consistent with the maximum and/or minimum established.

It will generally be found preferable to select a composition capable of delivering the desired release and/or pharmacokinetic profile when administered once daily.

Another embodiment of the invention provides a method for treating a catecholamine-mediated disease or disorder in a patient exhibiting a diurnal cycle of plasma concentration of a catecholamine, the method comprising
(1) establishing for the patient a diurnal time of peak plasma concentration of a catecholamine;
(2) identifying a daily administration time for antihypertensive medication; and
(3) administering clonidine once daily at the administration time in a dose of about 0.1 to about 2 mg/day, more typically about 0.1 to about 1 mg/day, to the patient in a form of a pharmaceutical composition comprising clonidine or a pharmaceutically acceptable salt or prodrug thereof and at least one pharmaceutically acceptable excipient; wherein the composition exhibits clonidine release properties providing, when so administered at a similar administration time and similar dose to a plurality of test subjects, a 24-hour profile of plasma clonidine concentration, averaged over the test subjects, exhibiting a peak that substantially coincides with or closely anticipates the peak in plasma catecholamine concentration established for the patient.

A diurnal time of peak plasma concentration of a catecholamine, for example norepinephrine, in the patient can be established by measuring plasma catecholamine levels using established laboratory techniques in blood samples collected from the patient at different times in a 24-hour cycle.

The daily administration time identified can be any time of day or night, but for most patients is most conveniently in the morning or evening. The daily administration time is not a precise clock time but should generally not vary by more than about 4 hours from day to day.

Upon establishing a time of peak plasma catecholamine concentration in the patient, and identifying a suitable administration time, a clonidine composition can be selected having release and/or pharmacokinetic properties consistent therewith, when administered at such a time, for example in the morning or evening.

Yet another embodiment of the invention provides a method for treating a catecholamine-mediated disease or disorder in a patient exhibiting a diurnal cycle of plasma concentration of a catecholamine, the method comprising
(1) establishing for the patient a diurnal time of peak plasma concentration or a diurnal period of sustained high plasma concentration of a catecholamine;
(2) selecting a composition comprising clonidine or a pharmaceutically acceptable salt or prodrug thereof and at least one pharmaceutically acceptable excipient, said composition exhibiting clonidine release properties providing, when administered to a plurality of test subjects, a 24-hour profile of plasma clonidine concentration having a peak or plateau; and
(3) administering the composition once daily in a clonidine dose of about 0.1 to about 2 mg/day, more typically about 0.1 to about 1 mg/day, to the patient, at a daily administration time selected such that the peak or plateau in plasma concentration of clonidine substantially coincides with or closely anticipates the peak or period of sustained high plasma concentration of catecholamine established for the patient.

A diurnal time of peak plasma concentration of a catecholamine, for example norepinephrine, in the patient can be established as described above.

If the diurnal cycle of plasma catecholamine concentration exhibits a strong diurnal peak, the composition selected should generally be one exhibiting a peak in 24-hour plasma clonidine concentration. A daily administration time for the composition is then selected such that the peak in plasma concentration of clonidine substantially coincides with or closely anticipates the peak plasma concentration of catecholamine established for the patient.

Alternatively, if the diurnal cycle of plasma catecholamine concentration does not exhibit a strong peak but a more extended plateau, including a plateau that is maintained for substantially the entire 24-hour cycle, the composition selected should generally be one exhibiting a period of sustained high plasma concentration of clonidine. A daily administration time for such a composition is then selected such that the period of high plasma concentration of clonidine substantially coincides with the plateau in plasma concentration of catecholamine established for the patient.

For example, certain people do not exhibit a substantial (e.g., greater than about 10%) nocturnal dip in plasma catecholamine level. This is referred to as a “non-dipping” cycle; for patients exhibiting a non-dipping cycle it can be important to select a time of administration of the clonidine composition that provides a relatively high plasma concentration of clonidine throughout the night.

The daily administration time selected can be any time of day or night, but for most patients is most conveniently in the morning or evening, and again is not a precise clock time but should generally not vary by more than about 4 hours from day to day.

Variants and illustrative modalities of the clonidine administering step in each of these embodiments are as described hereinabove.

According to any method described hereinabove wherein the disease or disorder treated comprises a cardiovascular condition, such method optionally further comprises administering to the patient one or more additional cardiovascular agents. An additional cardiovascular agent can be administered, for example, in combination or adjunctive therapy with the clonidine. Any cardiovascular agent appropriate to the particular condition being treated can be used. In one embodiment one or more additional cardiovascular agents are administered in combination with the clonidine, such additional agents being selected from antihypertensive drugs, antihyperlipidemic drugs, anticoagulants, antiarrhythmics, antianginal drugs, antiplatelet drugs, anti-inflammatories, nutritionals and combinations thereof.

Antihypertensive drugs that can be useful as additional cardiovascular agents according to the present embodiment include diuretics, angiotensin converting enzyme (ACE) inhibitors, angiotensin II receptor blockers, beta-adrenergic receptor blockers, calcium channel blockers, direct vasodilators, alpha-1-adrenergic receptor blockers, central alpha-2-adrenergic receptor blockers (other than clonidine) and aldosterone receptor antagonists.

Suitable diuretics illustratively and without limitation include chlorothiazide, chlorthalidone, hydrochlorothiazide, indapamide, metolazone, polythiazide, bumetanide, furosemide and torsemide.

Suitable ACE inhibitors illustratively and without limitation include benazepril, captopril, enalapril, fosinopril, lisinopril, moexipril, perindopril, quinapril, ramipril and trandolapril.

Suitable angiotensin II receptor blockers illustratively and without limitation include candesartan, eprosartan, irbesartan, losartan, olmesartan, tasosartan, telmisartan and valsartan.

Suitable beta-adrenergic receptor blockers illustratively and without limitation include acebutolol, atenolol, betaxolol, bisoprolol, carvedilol, labetalol, metoprolol, nadolol, penbutolol, pindolol, propranolol, sotalol and timolol

Suitable calcium channel blockers illustratively and without limitation include amlodipine, diltiazem, felodipine, isradipine, nicardipine, nifedipine, nisoldipine and verapamil.

Suitable direct vasodilators illustratively and without limitation include hydralazine and minoxidil.

Suitable alpha-1-adrenergic receptor blockers illustratively and without limitation include carvedilol, doxazosin, labetalol, prazosin and terazosin.

Suitable central alpha-2-adrenergic receptor agonists illustratively and without limitation include guanabenz, guanfacine and moxonidine.

Suitable aldosterone receptor antagonists illustratively and without limitation include canrenone, eplerenone and spironolactone.

Antihyperlipidemic drugs that can be useful as additional cardiovascular agents according to the present embodiment include, illustratively and without limitation, colesevelam, colestipol, clofibrate, fenofibrate, ezetimibe and 3-hydroxy-3-methylglutaryl-coenzyme A (HMG CoA) reductase inhibitors such as atorvastatin, cerivastatin, fluvastatin, lovastatin, pravastatin and simvastatin.

Anticoagulants that can be useful as additional cardiovascular agents according to the present embodiment include, illustratively and without limitation, acenocoumarol, anisindione, clorindione, dicumarol, heparin and warfarin.

Antiarrhythmics that can be useful as additional cardiovascular agents according to the present embodiment include, illustratively and without limitation, adenosine, digoxin, flecamide and propafenone.

Antianginal drugs that can be useful as additional cardiovascular agents according to the present embodiment include, illustratively and without limitation, acebutolol, amlodipine, atenolol, diltiazem, isosorbide dinitrate, isradipine, metoprolol, nadolol, nicardipine, nifedipine, nitroglycerin, pindolol, propranolol, ranolazine, sotalol, timolol and verapamil.

Antiplatelet drugs that can be useful as additional cardiovascular agents according to the present embodiment include, illustratively and without limitation, abciximab, anagrelide, aspirin, clopidogrel, dipyridamole, eptifibatide, iloprost, ticlodipine and tirofiban.

Anti-inflammatories that can be useful as additional cardiovascular agents according to the present embodiment include steroidal and nonsteroidal anti-inflammatories.

Suitable steroidal anti-inflammatory drugs include, illustratively and without limitation, beclomethasone, budesonide, dexamethasone, flunisolide, fluticasone, hydrocortisone, methylprednisolone, prednisolone, prednisone and triamcinolone.

Suitable nonsteroidal anti-inflammatory drugs (NSAIDs), including selective COX-2 inhibitors, include, illustratively and without limitation, aspirin, carprofen, celecoxib, diclofenac, diflunisal, etanercept, etodolac, fenoprofen, flurbiprofen, ibuprofen, indomethacin, infliximab, ketoprofen, ketorolac, meloxicam, naproxen, nimesulide, olsalazine, oxaprozin, salsalate, sulfasalazine and sulindac.

Nutritionals that can be useful as additional cardiovascular agents according to the present embodiment include, illustratively and without limitation, folate, omega-3 fatty acids and phytosterols.

Selection of a suitable clonidine composition for the individual patient can be made from a set of pre-formulated compositions. Alternatively, the clinician can prescribe a composition having a particular, even unique, combination of immediate, extended and/or delayed release components that is customized to the individual patient, and the composition can be prepared, for example by a pharmacist, by combining (e.g., mixing or blending) such components in a weight ratio as defined by the prescription.

As well as selecting a suitable composition, tailoring of a once-daily oral clonidine therapy regimen to a patient's individual needs can include selection of a dose, or progression of doses; selection of a suitable time of day for administration; prescribing additional drugs to be used adjunctively or cotherapeutically with the clonidine; etc. The method optionally further comprises monitoring of therapeutic, e.g., antihypertensive, efficacy and incidence of adverse effects at suitable intervals to permit adjustment of the dose and/or change of the prescription to a composition having different release characteristics.

In addition to the therapeutic methods described hereinabove, the present invention provides a pharmaceutical composition useful according to such methods. The composition comprises clonidine or a pharmaceutically acceptable salt or prodrug thereof, and exhibits clonidine release properties, when orally administered to a plurality of test subjects once daily in an amount delivering a clonidine dose of about 0.1 to about 1 mg/day, that provide a 24-hour profile of plasma clonidine concentration, averaged over the test subjects, that does not substantially or protractedly fall below about 0.2 ng/ml and does not substantially or protractedly exceed about 1 ng/ml.

Variants and illustrative modalities of the clonidine form (free base, salt and/or prodrug), dose, release properties and plasma clonidine concentration profile are as described hereinabove.

A composition of the invention is most conveniently presented as a discrete solid orally deliverable dosage form, such as a tablet or capsule.

As mentioned above, the composition according to certain embodiments comprises (a) a first formulation component comprising clonidine exhibiting a first release profile, for example an immediate release profile, and (b) a second formulation component comprising clonidine exhibiting a second release profile that is different from the first release profile, for example an extended and/or delayed release profile. In the case of a discrete solid dosage form, the first and second formulation components can be more or less intimately co-mixed or blended, or alternatively can form spatially distinct zones of the dosage form.

An illustrative composition has spatially distinct zones comprising a core and a mantle surrounding the core. In such a composition, the mantle can comprise clonidine exhibiting an immediate release profile and the core can comprise clonidine exhibiting an extended and/or delayed release profile.

Another illustrative composition has spatially distinct zones comprising at least two layers. An example of such a composition is a bilayer tablet, wherein one layer comprises clonidine exhibiting an immediate release profile and the other layer comprises clonidine exhibiting an extended and/or delayed release profile.

In one embodiment, the composition comprises first and second formulation components as described above, which comprise particles of a first and second kind respectively. For example, the particles of the first kind can exhibit an immediate release profile and the particles of the second kind can exhibit an extended and/or delayed release profile. Illustratively, the particles of the second kind comprise a core comprising the clonidine and an extended and/or delayed release coating surrounding the core.

Any formulation technology providing extended and/or delayed release can be employed. Description of a particular embodiment below is provided for illustrative purposes, and is not limiting to the scope of the invention.

An orally deliverable pharmaceutical dosage form illustrative of the invention comprises
(a) zero to about 50% by weight of particles of a first kind comprising clonidine or a pharmaceutically acceptable salt or prodrug thereof in a first amount, said particles of the first kind (i) each comprising an inert substrate having a clonidine-containing layer thereon, and (ii) exhibiting a clonidine immediate release profile; and
(b) about 50% to 100% by weight of particles of a second kind comprising clonidine or a pharmaceutically acceptable salt or prodrug thereof in a second amount, said particles of the second kind (i) each comprising a particle of the first kind, additionally having a coating that comprises a dissolution modifying system comprising a film forming agent and a plasticizer, and (ii) exhibiting a clonidine extended release profile.

The clonidine or salt or prodrug thereof is present in the dosage form in a total clonidine equivalent amount of about 0.1 to about 2 mg, for example about 0.1 to about 1 mg. Three features:
(i) weight ratio of particles of the first kind to particles of the second kind;
(ii) the first and second amounts of clonidine; and
(iii) the particular dissolution modifying system present in the coating of the particles of the second kind, including coating weight and particular film forming agent(s) and amount(s) thereof;
   operate to provide clonidine release properties consistent with those specified hereinabove.

Optionally, but preferably, the particles are enclosed in a hard or soft capsule shell to provide a discrete dosage form containing a unit dosage amount of clonidine.

Illustratively, the dosage form can be prepared as described in above-cited U.S. Pat. No. 5,133,974, which is incorporated herein by reference in its entirety. One of ordinary skill in the art can, based on the present specification and without undue experimentation, adapt the formulations described in U.S. Pat. No. 5,133,974 by adjustment of the three features listed above, to provide a release profile suitable for clonidine therapy according to a method of the present invention.

In one embodiment, such a dosage form has release properties providing, when the dosage form is orally administered one to three times daily to a plurality of test subjects, a 24-hour profile of plasma clonidine concentration, averaged over the test subjects, that does not substantially or protractedly fall below about 0.2 ng/ml (for example about 0.25, about 0.3, about 0.35, about 0.4, about 0.45, about 0.5, about 0.55 or about 0.6 ng/ml), and exhibits a peak concentration that does not substantially or protractedly exceed about 2.5 ng/ml (for example about 2.25, about 2, about 1.75, about 1.5, about 1.25, about 1, about 0.9, about 0.8, about 0.7 or about 0.6 ng/ml). Typically, for most subjects, it will be found suitable to provide a peak concentration that does not substantially or protractedly exceed about 1 ng/ml. Typically, it will be found suitable to provide a 24-hour profile of plasma clonidine concentration that does not substantially or protractedly fall below about one-fifth of the peak concentration. It will generally be found preferable to provide a dosage form as described above having release properties such that the desired plasma clonidine concentration profile is achieved with once daily administration.

All patents and publications cited herein are incorporated by reference into this application in their entirety.

The words “comprise”, “comprises”, and “comprising” are to be interpreted inclusively rather than exclusively.

What is claimed is:

1. A method for treating a disease or disorder for which clonidine is indicated in a patient, comprising orally administering clonidine once daily in a dose of about 0.1 to about 2 mg to the patient in a form of a pharmaceutical composition comprising clonidine or a pharmaceutically acceptable salt or prodrug thereof and at least one pharmaceutically acceptable excipient; wherein the composition exhibits clonidine release properties providing, when administered at a similar dose to a plurality of test subjects, a 24-hour profile of plasma clonidine concentration, averaged over the test subjects, that does not substantially or protractedly fall below about 0.2 ng/ml and does not substantially or protractedly exceed about 1 ng/ml.

2. The method of claim 1, wherein the daily dose of clonidine is about 0.1 to about 1 mg.

3. The method of claim 1, wherein the 24-hour plasma clonidine concentration profile, averaged over the test subjects, does not substantially or protractedly exceed about 0.8 ng/ml.

4. The method of claim 1, wherein the 24-hour plasma clonidine concentration profile, averaged over the test subjects, does not substantially or protractedly fall below about 0.2 ng/ml.

5. The method of claim 1, wherein the 24-hour plasma clonidine concentration profile, averaged over the test subjects, does not substantially or protractedly fall below about 0.4 ng/ml.

6. The method of claim 1, wherein the 24-hour plasma clonidine concentration profile, averaged over the test subjects, does not substantially or protractedly fall below about 0.4 ng/ml and does not substantially or protractedly exceed about 0.8 ng/ml.

7. The method of claim 1, wherein the composition releases clonidine over a period of at least about 8 hours as measured in a standard in vitro dissolution assay.

8. The method of claim 1, wherein the disease or disorder is selected from the group consisting of hypertension, arrhythmia, myocardial ischemia, atrial fibrillation, congestive heart failure, allodynia, hyperalgesia, neuropathic pain, cancer pain, cluster headache, chronic headache, migraine, postoperative pain, spinal cord injury pain, akathisia, restless legs syndrome, peripheral neuropathy, neuralgia, orofacial pain, diabetic gastroparesis, chronic memory disorders, hypertonia, hyperkinetic movement disorders, Tourette's syndrome, substance withdrawal, attention deficit hyperactivity disorder, manic states, behavioral disorders related to encephalopathy, bipolar disorder, narcolepsy, post-traumatic stress disorder, schizophrenia, sleep disorders, social phobia, hyperthyroidism, growth delay, excessive sweating, hot flashes, trichorrhexis nodosa, and combinations thereof.

9. The method of claim 1, wherein the disease or disorder comprises a cardiovascular condition.

10. The method of claim 1, wherein the disease or disorder is mediated by a catecholamine.

11. The method of claim 10, wherein the patient exhibits a diurnal cycle of plasma concentration of the catecholamine having at least one diurnal peak, wherein the composition is administered at a daily administration time, and wherein the 24-hour plasma clonidine concentration profile, averaged over the test subjects, exhibits a peak that, when the composition is administered at said daily administration time, substantially coincides with or closely anticipates the at least one diurnal peak in plasma catecholamine concentration in the patient.

12. The method of claim 11, wherein the composition is administered to the patient about 4 to about 16 hours prior to the at least one diurnal peak in plasma catecholamine concentration.

13. A method for treating a catecholamine-mediated disease or disorder in a patient, comprising
(1) establishing, for the patient, at least one of (a) a maximum plasma concentration of clonidine associated with an unacceptable side effect and (b) a minimum plasma concentration of clonidine associated with an unacceptable rebound or augmentation effect; and
(2) administering clonidine one to three times daily in a dose of about 0.1 to about 2 mg/day to the patient in a form of a pharmaceutical composition comprising clonidine or a pharmaceutically acceptable salt or prodrug thereof and at least one pharmaceutically acceptable excipient; wherein the composition exhibits clonidine release properties providing, when so administered at a similar dose to a plurality of test subjects, a 24-hour profile of plasma clonidine concentration, averaged over the test subjects, that does not substantially or protractedly fall below the minimum or does not substantially or protractedly exceed the maximum plasma concentration of clonidine established for the patient.

14. The method of claim 13, wherein both (a) a maximum plasma concentration of clonidine associated with an unacceptable side effect and (b) a minimum plasma concentration of clonidine associated with an unacceptable rebound or augmentation effect are established for the patient; and wherein the 24-hour profile of plasma clonidine concentration provided by administration of the concentration, averaged over the test subjects, neither substantially or protractedly falls below the minimum nor substantially or protractedly exceeds the maximum plasma concentration of clonidine established for the patient.

15. The method of claim 13, further comprising establishing for the patient a diurnal time of peak plasma concentration of the catecholamine; wherein the 24-hour profile of plasma clonidine concentration provided by administration of the concentration, averaged over the test subjects, exhibits a peak that substantially coincides with or closely anticipates the peak in plasma catecholamine concentration established for the patient.

16. The method of claim 13, wherein the composition is administered once daily.

17. A method for treating a catecholamine-mediated disease or disorder in a patient exhibiting a diurnal cycle of plasma concentration of a catecholamine, the method comprising
(1) establishing for the patient a diurnal time of peak plasma concentration of a catecholamine;
(2) identifying a daily administration time for antihypertensive medication; and
(3) administering clonidine once daily at the administration time in a dose of about 0.1 to about 2 mg/day to the patient in a form of a pharmaceutical composition comprising clonidine or a pharmaceutically acceptable salt or prodrug thereof and at least one pharmaceutically acceptable excipient; wherein the composition exhibits clonidine release properties providing, when so administered at a similar administration time and similar dose to a plurality of test subjects, a 24-hour profile of plasma clonidine concentration, averaged over the test subjects, exhibiting a peak that substantially coincides with or closely anticipates the peak in plasma catecholamine concentration established for the patient.

18. A method for treating a catecholamine-mediated disease or disorder in a patient exhibiting a diurnal cycle of plasma concentration of a catecholamine, the method comprising
(1) establishing for the patient a diurnal time of peak plasma concentration or a diurnal period of sustained high plasma concentration of a catecholamine;
(2) selecting a composition comprising clonidine or a pharmaceutically acceptable salt or prodrug thereof and at least one pharmaceutically acceptable excipient, said composition exhibiting clonidine release properties providing, when administered to a plurality of test subjects, a 24-hour profile of plasma clonidine concentration having a peak or plateau; and
(3) administering the composition once daily in a clonidine dose of about 0.1 to about 2 mg/day to the patient, at a daily administration time selected such that the peak or plateau in plasma concentration of clonidine substantially coincides with or closely anticipates the peak or period of sustained high plasma concentration of catecholamine established for the patient.

19. The method of claim 18, wherein the catecholamine-mediated disease or disorder is hypertension or a condition or event arising therefrom.

20. The method of claim 18, wherein the diurnal cycle is non-dipping.

21. The method of claim 20, wherein the non-dipping cycle is associated with diabetes and/or kidney disease in the patient.

22. A pharmaceutical composition comprising clonidine or a pharmaceutically acceptable salt or prodrug thereof, that when orally administered to a plurality of test subjects once daily in an amount delivering a clonidine dose of about 0.1 to about 2 mg/day, exhibits clonidine release properties providing a 24-hour profile of plasma clonidine concentration, averaged over the test subjects, that does not substantially or protractedly fall below about 0.2 ng/ml and does not substantially or protractedly exceed about 1 ng/ml.

23. The composition of claim 22, wherein the 24-hour plasma clonidine concentration profile, averaged over the test subjects, does not substantially or protractedly exceed about 0.8 ng/ml.

24. The composition of claim 22, wherein the 24-hour plasma clonidine concentration profile, averaged over the test subjects, does not substantially or protractedly fall below about 0.2 ng/ml.

25. The composition of claim 22, wherein the 24-hour plasma clonidine concentration profile, averaged over the test subjects, does not substantially or protractedly fall below about 0.4 ng/ml.

26. The composition of claim 22, wherein the 24-hour plasma clonidine concentration profile, averaged over the test subjects, does not substantially or protractedly fall below about 0.4 ng/ml and does not substantially or protractedly exceed about 0.8 ng/ml.

27. The composition of claim 22, that releases clonidine over a period of at least about 8 hours as measured in a standard in vitro dissolution assay.

28. The composition of claim 22, in a form of a discrete solid orally deliverable dosage form.

29. The composition of claim 28, wherein the dosage form is a tablet or capsule.

30. The composition of claim 22, that exhibits extended or delayed release in a standard in vitro dissolution assay.

31. The composition of claim 22, comprising (a) a first formulation component comprising clonidine exhibiting a first release profile, and (b) a second formulation component comprising clonidine exhibiting a second release profile that is different from the first release profile.

32. The composition of claim 31, wherein the first release profile is an immediate release profile and the second release profile is an extended and/or delayed release profile.

33. The composition of claim 31, wherein the dosage form is a discrete solid dosage form and the first and second formulation components form spatially distinct zones of the dosage form.

34. The composition of claim 33, wherein the spatially distinct zones comprise a core and a mantle surrounding the core.

35. The composition of claim 34, wherein the mantle comprises clonidine exhibiting an immediate release profile and the core comprises clonidine exhibiting an extended and/or delayed release profile.

36. The composition of claim 33, wherein the spatially distinct zones comprise at least two layers.

37. The composition of claim 36, in a form of a bilayer tablet.

38. The composition of claim 31, wherein the first and second formulation components comprise particles of a first and second kind respectively.

39. The composition of claim 38, wherein the particles of the first kind exhibit an immediate release profile and the particles of the second kind exhibit an extended and/or delayed release profile.

40. The composition of claim 39, wherein the particles of the second kind comprise a core comprising the clonidine and an extended and/or delayed release coating surrounding the core.

41. The composition of claim 22, wherein the 24-hour profile of plasma clonidine concentration exhibits a peak about 4 to about 16 hours after administration.

42. An orally deliverable pharmaceutical dosage form comprising
(a) zero to about 50% by weight of particles of a first kind comprising clonidine or a pharmaceutically acceptable salt or prodrug thereof in a first amount, said particles of the first kind (i) each comprising an inert substrate having a clonidine-containing layer thereon, and (ii) exhibiting a clonidine immediate release profile; and
(b) about 50% to 100% by weight of particles of a second kind comprising clonidine or a pharmaceutically acceptable salt or prodrug thereof in a second amount, said particles of the second kind (i) each comprising a particle of the first kind, additionally having a coating that comprises a dissolution modifying system comprising a film forming agent and a plasticizer, and (ii) exhibiting a clonidine extended release profile;
wherein the clonidine or salt or prodrug thereof is present in a total clonidine equivalent amount of about 0.1 to about 2 mg; and wherein the weight ratio of particles of the first kind to particles of the second kind, said first and second amounts, and said dissolution modifying system operate to provide, when the dosage form is orally administered one to three times daily to a plurality of test subjects, a 24-hour profile of plasma clonidine concentration, averaged over the test subjects, that does not substantially or protractedly fall below about 0.2 ng/ml and exhibits a peak concentration that does not substantially or protractedly exceed about 2.5 ng/ml.

43. The dosage form of claim 42, wherein the 24-hour profile of plasma clonidine concentration exhibits a peak about 4 to about 16 hours after administration.

44. The dosage form of claim 42, wherein the clonidine or salt or prodrug thereof is present in a total clonidine equivalent amount of about 0.1 to about 1 mg.

45. The dosage form of claim 42, wherein said 24-hour profile of plasma clonidine concentration is achieved with once daily administration.

46. The dosage form of claim 42, wherein the peak concentration does not substantially or protractedly exceed about 1 ng/ml.

47. The dosage form of claim 42, wherein the 24-hour profile of plasma clonidine concentration, averaged over the test subjects, does not substantially or protractedly fall below about one-fifth of the peak concentration.
